(19) United States
(12) Patent Application Publication
(10) Pub. No.: US 2012/0165598 A1
(43) Pub. Date: Jun. 28, 2012

(54) DEVICE FOR IMPROVING AN INDIVIDUAL'S PHYSICAL PERFORMANCE AND ABILITY TO MAINTAIN BALANCE, AND METHOD FOR MANUFACTURING THEREOF

(76) Inventors: David Scalco, Cartigliano (VI) (IT); Alberto Olivetto, Tombolo(PD) (IT)

(21) Appl. No.: 13/254,578

(22) PCT Filed: Feb. 26, 2010
(86) PCT No.: PCT/IB10/50839

§ 371 Date: Mar. 15, 2012

(30) Foreign Application Priority Data
Mar. 2, 2009 (IT) PD2009A000034

(51) Int. Cl. A61M 21/00

(52) U.S. Cl. 600/26

(57) ABSTRACT

A device (1) for improving an individual's physical performance and ability to maintain balance, which is suitable for being placed close to a portion of the individual's skin, comprises at least one metal body (2, 2′) which is provided with a first metal layer (4, 4′), and at least one second metal layer (5, 7) that is based on a metal selected from ruthenium, rhodium and platinum and is at least partially superimposed on the first layer (4, 4′).

### TECHNICAL FIELD

The present invention relates to a device for improving an individual's physical performance and ability to maintain balance, comprising the features set out in the preamble of claim 1.

The invention further relates to a garment or an accessory, or even a jewel, provided with a device for improving an individual's physical performance and ability to maintain balance.

The invention also relates to a method of producing a device for improving an individual's physical performance having the features set out in claim 17.

### BACKGROUND ART

Wristbands or similar devices which, when worn, exert some pressure on predetermined known points of the body that are widely used in reflexology so as to stimulate reflexes which are sent to the user's brain are known.

The brain thus receives an improved flow of neurological information to which it responds by improving the body's functional capacities.

Moreover, the beneficial effects resulting from direct or indirect contact of some metals with some parts of the human body are known.

For example, it is known from RU2268707 to cure some painful conditions by applying metallic plasters in suitable application zones of a patient's body.

From CN1260216 it is known to treat encephalopathy by the application of magnetic elements to sensitive points that are also used in acupuncture.

However, there is scope for improvements in devices for improving an individual's performance and consequently in the improvements obtainable in the individual.

Moreover, some known devices may even have adverse effects on the patient's health or general physical condition.

### DESCRIPTION OF THE INVENTION

The problem underlying the present invention is to provide a device which improves an individual's physical performance and ability to maintain balance.

A further objective is to provide a device which reduces the effects of muscle fatigue in an individual, in particular in an athlete.

A further objective is to provide a device which reduces the effects of chronic fatigue syndrome.

A further objective is to provide a device which increases an individual's stability.

A further objective is to provide a device which increases the muscle power and hence the sports performance of an individual, an athlete, or an untrained individual, particularly in jumping, pedalling or running.

A further objective is to provide a device which is durable over time and which is not subject to wear and therefore retains its functions for a very prolonged period of time.

Yet another objective is to provide a device which improves an individual's general physical condition.

These problems are solved and these objectives are achieved by means of a device for improving an individual's physical performance and ability to maintain balance and a method for the production thereof, according to the appended claims.

### BRIEF DESCRIPTION OF THE DRAWINGS

The characteristics and the advantages of the invention will become clearer from the detailed description of a preferred embodiment thereof which is described by way of non-limiting example with reference to the appended drawings, in which:

FIG. 1 is a plan view of a device formed in accordance with the present invention;

FIGS. 2-8 are enlarged, truncated through sections variants of a device according to the invention;

FIGS. 9a-9c are graphs which show the IR radiation emission of a remote control in normal conditions of use (curve A) with a device of the invention fitted on the battery (curve B) and with a device of the invention fitted on the LED (curve C), for various wavelength ranges;

FIGS. 10a-10b are graphs which show the IR radiation emission of a cell phone when receiving a call in normal conditions of use (curve A′), with a device of the invention fitted on the battery (curve B′), and with a device of the invention fitted on the screen (curve C′), for various wavelength ranges;

FIGS. 11a and 11b are graphs which show the contraction power of an individual's pectoral muscles (FIG. 11a) and anterior deltoid muscle (FIG. 11b) with and without the device of the invention;

FIG. 12a-12b are graphs which show an individual's pedalling power (FIG. 12a) and force (FIG. 12b), respectively, with and without the device of the invention;

FIGS. 13a-13d are graphs which show, respectively, the jumping heights of a group of female basket-ball players (FIG. 13a), of a group of male basket-ball players (FIG. 13b), of a group of football players (FIG. 13c), and of a group of non-sporting individuals (FIG. 13d) with and without the device of the invention;

FIGS. 14a and 14b are graphs which show the jumping heights and the jumping power, respectively, of a football player with and without the device of the invention; and

FIG. 15a-15b are graphs which show the displacement of an individual's centre of gravity in a shooting test, without the device of the invention (FIG. 15a) and with the device of the invention (FIG. 15b).

### PREFERRED EMBODIMENT OF THE INVENTION

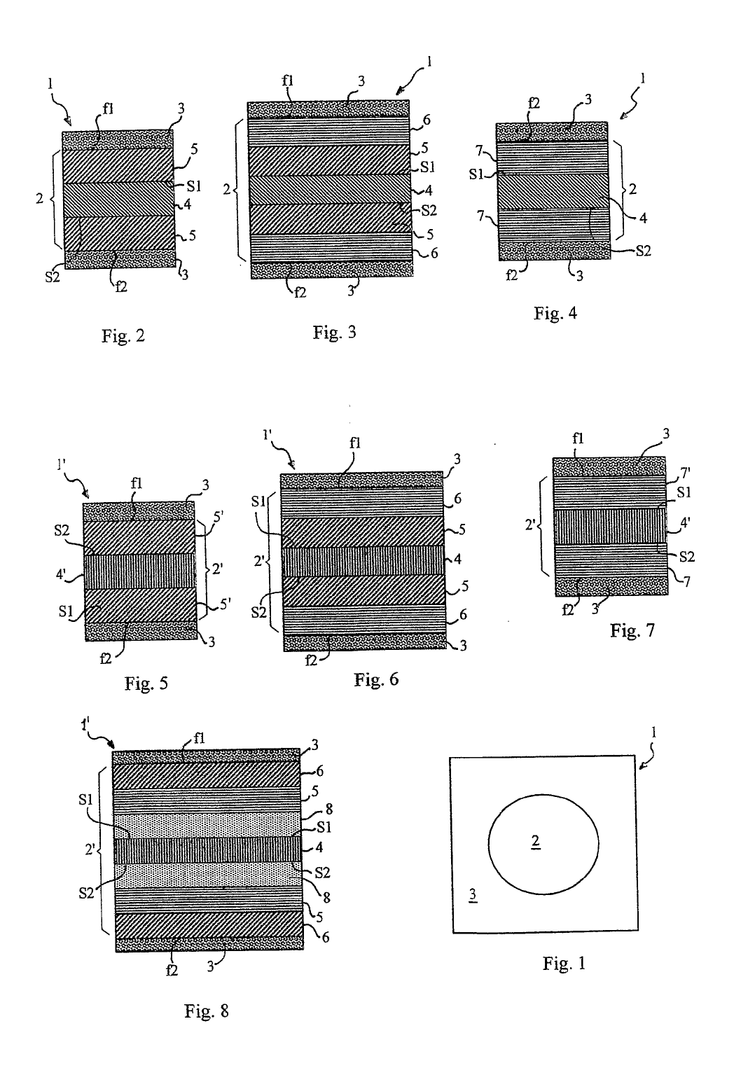

A device formed in accordance with the present invention for improving an individual's physical performance and ability to maintain balance is generally indicated 1 in FIGS. 1-8.

The device 1 can be positioned in direct or indirect contact with a portion of an individual's skin.

The device 1 may also be positioned beneath the individual's skin or may even be positioned some distance, preferably less than 3 cm, preferably between 0 and 2 cm, from a portion of the individual's skin.

The device 1 comprises a metal body 2 that is externally provided with a covering 3 that is made of insulating material and is formed so as to cover the metal body 2.

The covering 3 may be made of any insulating material suitable for contact with an individual's skin.

The covering 3 is preferably made of material which is anallergic in order not to trigger allergic reactions and which has an affinity for metals so that the covering 3 correctly adheres to the body 2.

The covering 3 may be made, for example, of polymer material such as PVC, polyurethane (PU), nylon, polypropylene, polyethylene (PE), high-density polyethylene (HDPE), PET or fibres derived therefrom, PTFE, or other plastics materials, or even silicone rubbers.

In other versions, the covering 3 may be made of an insulating material of natural origin, for example, rubber, natural gum, or latex. In other versions, the covering 3 may be made of a natural insulating fibre, for example, wool, cotton, hemp and the like. In this case, an adhesive film may be interposed between the metal body 2 and the covering 3 to improve the adhesion of the covering 3 to the metal body 2 and the covering 3 may also seal the metal body 2 from the exterior.

The use of polypropylene or of another biocompatible material enables a device suitable for subcutaneous application to be produced.

The insulating covering 3 is formed so as to cover at least one of the opposed main faces f1, f2 of the metal body 2 at least partially. In the versions shown, the insulating covering 3 covers both of the opposed main faces f1, f2 of the metal body 2 completely.

In one version of the invention, the covering 3 is fitted on the metal body 2 in a manner such that the metal body is sealed from the exterior, that is, in a manner such that the covering 3 forms a kind of pouch inside which the metal body 2 is inserted and which is then closed from the exterior.

The presence of the covering 3 partially or completely covering the metal body 2 affords many advantages.

The covering 3 of insulating material insulates the metal body 2 of the device 1 from the exterior, limiting any effects of external conditions on the device 1 and on its functional features.

Moreover, the presence of the covering 3 protects the metal body 2 of the device 1 from abrasion or wear of the material of the metal body 2, enabling the features of the device 2 to be kept almost unchanged over time.

In particular, the covering 3 greatly limits or reduces oxidative effects on the metal body 2.

A device which is durable and has constant performance over time is thus obtained.

The covering 3 is advantageously fitted in a manner such that the metal body 2 is under vacuum.

This further improves the performance of the device 1 and preserves even better its characteristics, properties and the integrity of the metal body 2 and hence of the device 1.

Oxidation of the metal body 2 is thus substantially eliminated, further increasing the stability and durability of the device 1 over time.

In other versions, not shown, the covering 3 may cover only one of the two opposed faces f1, f2 of the metal body 2 and/or may be provided so as to cover one or both of the two opposed faces f1, f2 partially.

When the covering 3 covers only one of the two main faces f1, f2 of the metal body 2, the device 1 will preferably be positioned in a manner such that the covering 3 faces a portion of the skin of the user of the device 1.

In the version shown, the device takes the form of a plate 1 having a quadrangular section in plan but plates 1 having any desired shape, for example, a circular shape, may be produced.

The metal body 2 preferably has a thickness of between about 0.2 mm and about 5 mm and preferably a circular shape with a diameter of between about 10 mm and about 40 mm.

Preferably, if the thickness of the metal body 2 is about 0.3 mm, the diameter is about 15 mm and, if the thickness of the metal body 2 is about 0.3 mm, the diameter is about 10 mm, that is, so that the metal body 2 comprises a predetermined overall mass of metal.

In the version of FIG. 2, the metal body 2 comprises a central layer 4 based on silver (Ag), covered by an intermediate layer 5 based on ruthenium (Ru), outside which the insulating covering 3 is provided.

The central layer 4 has a thickness of between about 0.2 and 0.5 mm, whereas the intermediate layer 5 has a thickness of between about 2 and 6 μm.

The intermediate layer 5 is shaped in a manner such as to cover both of the opposed main surfaces S1, S2 of the central layer 4.

In other versions, not shown, the intermediate layer 5 may cover only one of the two opposed surfaces S1, S2 of the central layer 4, optionally partially.

In the version of FIG. 3, the metal body 2 further comprises an outer layer 6 which is based on rhodium (Rh) and is arranged outside the intermediate, ruthenium-based layer 5 so as to be interposed between the latter and the insulating covering 3.

The outer layer 6 may be applied to both of the opposed main surfaces of the intermediate layer 5, as in the version shown, or to only one of the two surfaces and/or partially covering it/them.

The outer layer 6 has a thickness of between about 2 and 3μ.

In the version of FIG. 4, the metal body 2 comprises a central layer 4 based on silver (Ag), covered by a middle layer 7 based on rhodium (Rh), outside which the insulating covering 3 is fitted. The middle layer 7 has a thickness of between about 2 and 6 μm.

The middle layer 7 is also formed in a manner such as to cover both of the opposed main surfaces S1, S2 of the central layer 4.

In other versions, not shown, the middle layer 7 may cover only one of the two opposed surfaces S1, S2 of the central layer 4, optionally partially.

To form the outer, ruthenium-based layer, galvanic-bath ruthenium is used at a concentration of between 2 and 9 g/l.

To form the rhodium-based layers, galvanic-bath rhodium having a concentration of between 1 and 8 g/l is used.

These concentration values permit the use of very short contact times between the metal body 2 and the galvanic bath, that is, times of the order of seconds, and at the same time enables the above-indicated thicknesses to be obtained.

The contact time is between 2 and 10 seconds, although the metal body 2 may even be left in contact with the galvanic bath for 10 min.

In some embodiments, the metal body 2 is left in contact with the galvanic bath for 160 seconds.

By reducing the contact times, it is also possible to prevent blackening of the rhodium-based layer and, moreover, to use low voltages for the galvanic bath, that is, voltages of the order of 6-10 Volts, or up to 12 V.

Moreover, to achieve the thicknesses indicated above and at the same time to prevent the rhodium-based layer from tending to separate from the metal body 2 to which it is applied, the body is immersed in the galvanic bath for a desired period of time suitable for obtaining a rhodium-based layer of the desired thickness such as to prevent its detachment from the body 2 by repeatedly alternating the immersion steps with washing and degreasing steps, as explained further below.

The covering layer 3 of the device 1 may be formed in the following manner; the metal body 2 is interposed between two sheets of material suitable for constituting the covering 3 and having larger dimensions than the metal body 2 so that edges of the sheets project relative to the metal body 2.

The edges of the covering are then brought together by means of a press or similar device and are thus associated with one another permanently so as to give rise to a kind of closed pouch in which the metal body 2 is housed, surrounded by an outer edge formed of the covering material.

In some applications, the edges of the covering 3 are associated with one another by a suitable welding process, for example, by high-frequency welding.

In other applications, the covering 3 may be formed by moulding, for example, pressure or injection moulding or other moulding techniques selected on the basis of the material of which the covering 3 is to be formed which, in these cases, is preferably plastics material.

In the above-mentioned cases, the metal body 2 is inserted in a forming mould and the plastics material with which the covering 3 is to be formed is then introduced into the same mould.

Upon completion of the moulding process, an item in which the metal body 2 is incorporated inside the plastics material of the covering 3 is thus obtained.

This method may be used, for example, to obtain directly an item, for example, a ski boot, already provided with the device 1.

The material of the ski boot or, in general, of the item produced, will act as the insulating covering 3 for the metal body 2. The versions shown in FIGS. 5-7 correspond to those shown in FIGS. 2-4, respectively, and differ therefrom in that the central layer 4′ is based on gold (Au); the other features and components of the plate 1′ are identical to those of the plate shown in FIGS. 1-4 and are therefore not described in detail.

In the version of FIG. 5, the metal body 2′ comprises a central layer 4′ based on gold (Au), covered by an intermediate layer 5 based on ruthenium (Ru), outside which the polymer covering 3 is provided.

In the version of FIG. 6, the metal body 2′ also comprises an outer layer 6 based on rhodium (Rh), which is arranged outside the intermediate ruthenium-based layer 5 so as to be interposed between the layer 5 and the insulating covering 3.

In the version of FIG. 8, the metal body 2′ also comprises a layer 8 based on platinum (Pt) interposed between the central layer 4′ based on gold (Au) and the intermediate layer 5 based on ruthenium (Ru), as well as the outer layer 6 based on rhodium (Rh), and the insulating covering layer 3 outside the latter.

In the version of FIG. 7, the metal body 2 comprises a central layer 4′ based on gold (Au), covered by a layer 7 based on rhodium (Rh), outside which the insulating covering 3 is fitted.

In other versions, not shown, the central layer 4 of the metal body 2 may be based on platinum, copper, steel, aluminium, or any other desired metal.

The central layer 4 of the metal body 2 may also be made of an alloy based on silver, and/or gold, and/or platinum, and/or copper, and/or aluminium, or any other desired metal alloy.

High purity silver, for example, silver 999, may be used to form the central layer.

In versions not shown, the device 1 may have shapes other than a plate shape, for example, it may be in the form of wire, preferably having a diameter of between 0.2 mm and 1.2 cm.

The wire comprises the metal body 2, covered externally by the covering 3 of insulating material.

In view of the good workability of the metallic and non-metallic materials that are used in the production of the device 1, as will become clear from the following, there are no problems in producing wires even of limited thicknesses.

The Applicant has had tests carried out at the Department of Materials Engineering and Industrial Technology of Trento University to evaluate the interaction of the device 1 of the invention with electromagnetic radiation, in particular, to evaluate any transmission of infrared radiation by a device according to the invention.

In particular, the transmittance of the device of the invention in the infrared radiation range (800-25000 nm) was evaluated.

In the first case, a Jasco 660 FT-IR single-beam spectrophotometer was used and three spectra were acquired, one with an empty sample chamber (no device according to the invention) and another two relating to the transmission of the device in air and in a vacuum, respectively.

The three spectra could be superimposed without appreciable or significant variations and an absence of transmission of IR radiation by the device of the invention was therefore ascertained.

The tests were repeated in the N-IR (near infrared) wavelength range, that is, between 800 and 2500 nm, with the use of a high-resolution Jasco V-570 spectrophotometer characterized by a data pitch of 1 nm, by a scanning speed of 400 nm/min, and by a stray-light percentage of less than 0.03%.

The spectra acquired also showed a zero percentage of light transmitted, therefore demonstrating an absence of transmission of radiation by the device of the invention material also in this wavelength range considered.

The same tests were repeated with devices according to the invention having different sizes from one another, again confirming the absence of IR-radiation transmission by the device.

The tests were repeated over predetermined periods of time to check for any variations in transmission by a device according to the invention with the selection of a period of time of 300 seconds with a scanning interval of 20 seconds.

These latter measurements permitted the conclusion that there were no variations over time in the signal picked up, the values of which always fell within the limits of sensitivity of the instrument, that the dimensions of the device had no effect on its physical properties and, therefore, that a device formed in accordance with the invention does not transmit IR radiation within the wavelength range examined (800-25000 nm).

The devices of the invention were also subjected to thermal treatments with heating and cooling to 70° C. and to −20° C., respectively, to check for possible activation of transmissive processes.

The tests described above were also repeated on devices of the invention subjected to the above-mentioned treatments, confirming the to results obtained above, that is, no transmission in the infrared range (800-25000 nm).

The measurements taken showed that the devices of the invention transmits no radiation in the infrared (IR) range or in the near infrared (NEAR-IR) range, that is, over the entire wavelength range between 800 and 25000 nm.

Any interaction of a device according to the invention with instruments such as a conventional remote control and a cell phone, which behave as IR radiation emitters when in operation, was then investigated.

In the case of the remote control, the measurement procedure consisted in working in air, always with the primary beam intercepted, and hence collecting only that which was transmitted by the sample under test, and in positioning the remote control inside the sample chamber so as to ensure a fixed and repeatable measurement system.

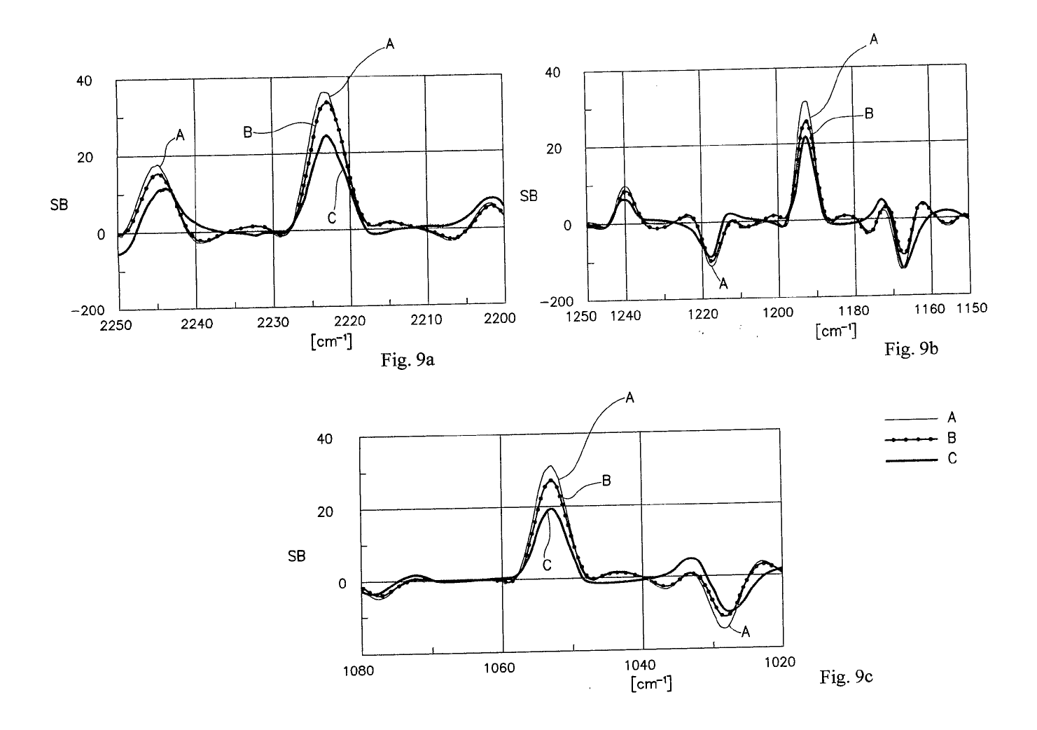

The results of the above-mentioned tests are given in the graphs of FIGS. 9a-9c which are subdivided by wavelength: FIG. 9a for wavelengths between 2200 and 2250 cm⁻¹, FIG. 9b for wavelengths between 1150 and 1250 cm⁻¹, and FIG. 9c for wavelengths between 102 and 108 cm⁻¹.

A first spectrum relating to the radiation emitted by the remote control whilst a key thereof was depressed (curve A), and two subsequent spectra again relating to the emission of the remote control, were acquired, naturally whilst keeping the same key depressed to prevent fluctuations in signal intensity, but after placing a device according to the invention on the battery of the remote control (curve C), and beside the LED on the front of the remote control (curve B), respectively.

Analysis of the results obtained shows that the spectra are superimposable and the presence of the device according to the invention did not therefore introduce new emission peaks and also confirms the measurements taken previously; however, in the region of three greater-intensity peaks at 2222 cm⁻¹ (FIG. 9a), 1192 cm⁻¹ (FIG. 9b), 1053 cm⁻¹ (FIG. 9c), the emission intensity decreased upon changing from operation of the remote control alone to operation with the presence of the device according to the invention. In particular, the greater decrease related to the situation in which the device according to the invention was on the battery of the remote control (curve C).

From an analysis of the graphs of FIGS. 9a-9c it can be seen that the emission spectrum of the remote control has a reduction in maximum intensity (peaks) and a displacement of the maximum intensity towards higher wavelengths and hence towards lower frequencies when a device according to the invention is associated with the remote control, this reduction being greater when the device of the invention is placed on the battery of the remote control (curve C).

Measurements were also taken to check for any influence exerted by a device according to the invention on the radiation emitted by a cell phone during the receipt of a call.

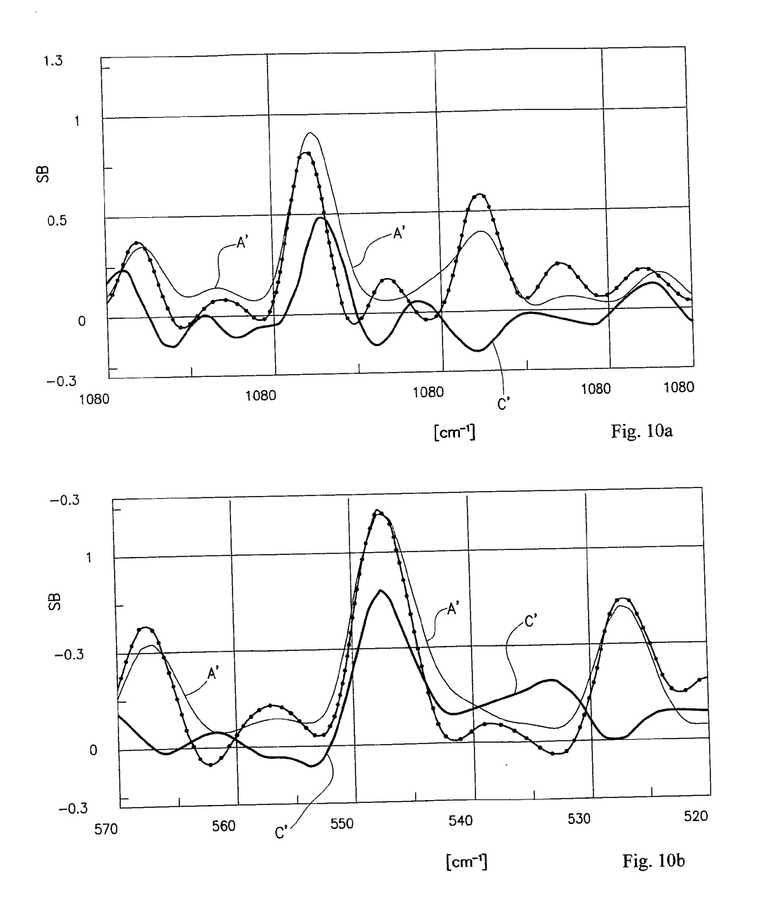

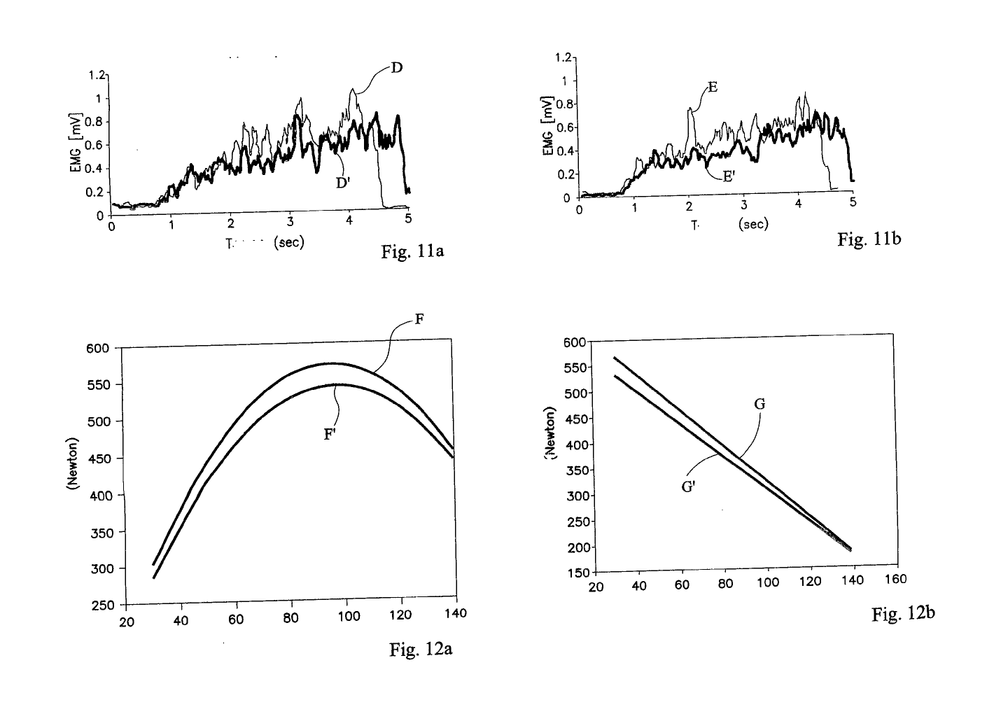

The IR spectra recorded are given in the graph of FIGS. 10a and 10b for different wavelength ranges: FIG. 10a for wavelengths between 1050 and 1120 cm⁻¹; FIG. 10b for wavelengths between 520 and 570 cm⁻¹.

In the above-mentioned graphs, the curves A′ relate to the spectra emitted by the cell phone during the receipt of a call, without the device of the invention, whereas the spectra of the curves B′ and C′ relate to cases in which a device according to the invention was positioned on the screen (curve B′) and on the battery (curve C′) of the same cell phone, respectively, again during the receipt of a call.

It is clear from the graphs of FIGS. 10a and 10b that a conventional cell phone is actually an emitter of IR radiation, although of low intensity, with two maximum peaks at 1093 and 547 cm⁻¹.

As with the tests carried out with the remote control, the presence of a device according to the invention on the screen and, to a greater extent, on the battery, again led to a reduction in the intensity of the radiation emitted.

The results of the tests reported above permit the conclusion that the application of a device of the invention to the battery of a remote control or of a cell phone leads to a reduction in the maximum intensities of the radiation emitted.

The devices of the invention therefore interact with devices emitting IR radiation, affecting emission intensity.

The results reported above can be improved further, in the sense of a greater absorption of infrared radiation, when a wire produced in accordance with the invention, that is, with a metal core and an outer covering of insulating material, is applied to a substantial portion of the periphery of a device such as a cell phone.

The Applicant has performed clinical tests to evaluate the type of effect of devices formed in accordance with the invention on an individual's body.

The above-mentioned tests were carried out by the Istituto di Semeiotica Biofisica Quantistica (Institute of Quantistic Biophysical Semeiotics) and showed that a device according to the invention has positive effects on an individual's organism within very short periods of time after its application in the vicinity of the individual's body.

The device of the invention acts as a biochemical catalyst, in fact accelerating the biochemical reactions which take place within the individual's organism.

The device of the invention also acts as an enzymatic activator, activates the cell membrane, favouring its exchanges, and cooperates in the breakdown of macromolecules.

The device of the invention acts as a histangio-protector, that is, it enables cell respiration to be normalized, if it is altered, within one hour of application and then, within three hours, activates histangic oxygenation bringing it to maximum values, improving it, even if it is compromised, for example, in the case of CAEMH (congenital acidosic enzymo-metabolic histangiopathy) raising free endocellular energy by 50%.

The above-mentioned positive effects were not accompanied by increase in free radicals by activation of mitochondrial respiration, by virtue also of increased synthesis of CoQ10 (coenzyme Q10) and of the significant improvement in the microcirculation of the organism, which was shown by a duration of the disappearance of the aspecific gastric reflex of a few seconds. Tests carried out showed that a device 1 according to the invention improved the ability of an individual who used it to maintain balance.

Tests carried out by the Applicant have also shown that a device 1 according to the invention improves the physical performance and physical strength of an individual who uses it.

In particular, as a result of these tests, it has been shown that the device of the invention increases physical strength and reduces the fatigue of an individual who uses it.

During a physical effort exceeding a predetermined work load (the metabolic threshold), the muscles produce a certain amount of lactic acid which flows into the blood to be reconverted into glucose.

During intense and prolonged physical exercise, the muscles produce more lactic acid than the organism can metabolize; in this case, the concentration of lactic acid in the blood increases to the point at which it can no longer be dissipated at the level of the active muscles.

The known effects of fatigue, sometimes accompanied by burning, and subsequent local incapacity for exertion thus arise since the lactic acid causes the pH to reduce locally and leads, at certain concentrations, to inhibition of the production of energy by the use of carbohydrates. This results in stoppage of muscular contraction.

With the use of a device according to the invention and when an individual was subjected to exertion, the levels of lactic acid in the blood measured for a given effort were considerably less than those measured in the absence of the device of the invention.

The device was positioned in direct or indirect contact with a portion of an individual's skin or even under the skin.

The differences between the lactic acid values found for a given effort in the presence and in the absence of the device were significant and could not be attributed to statistical errors or to typical test variances.

The device according to the invention thus enabled the dissipation of muscular lactic acid produced during physical exercise to be altered, reducing the lactic acid that was present in the muscles and thus improving the physical performance of the individuals using the device.

The concentration of lactic acid in the blood reflects the dynamic equilibrium between the production and the dissipation thereof.

Approximately 75-80% of the lactic acid produced during exercise is metabolized by catabolic means (oxidation to CO₂) and the remainder is metabolized by anabolic means (gluconeogenesis) or is eliminated by urinary excretion and perspiration.

Muscles, particularly those that are subjected to training, not only produce lactic acid but are also capable of using it and dissipation increases with exercise in proportion to metabolic intensity.

The reduction in the amount of lactic acid found thus permits the assumption that the usage of lactic acid by the muscle, and hence its dissipation, were increased.

The device of the invention thus increases an individual's resistance to exertion.

The Applicant has performed tests to analyze muscular activation in a maximum voluntary contraction on the same individual with and without the device of the invention; the results obtained are given in the graphs of FIGS. 11a and 11b, respectively.

A surface electromyography system (Biovision®) operating at 1000 Hz was used.

The individual was subjected to a maximum voluntary contraction test (MVC) for two muscles: pectoral and anterior deltoid, in the presence, curves D and E, or in the absence, curves D′ and E′, of a device according to the invention. This was in order to evaluate the effect of the devices of the invention on the performance of an exercising individual requiring a high force production.

As is easily seen from an analysis of the graphs of FIGS. 11a-11b, the tracings relating to the use of the devices of the invention are greater both in the peaks and in the total area, for a given time considered. This means that, if the tension required is maximum, the devices act by reinforcing the motor unit recruitment mechanisms (Henneman's principle) and the patient manages to produce more force.

With the use of the devices of the invention, there was an increase in activation intensity of +23% for the pectoral and +26% for the anterior deltoid. The devices of the invention “liberate” the neuromuscular activation integration mechanisms, inducing an increase in the recruitment of the motor units with a consequent greater production of force.

Tests were carried out to evaluate the power produced by an athlete (a woman cyclist) pedalling with and without the device of the invention. The Porter® Cyclomulino was used, which enables the power produced by the athlete to be evaluated by means of Kisciotte® software.

The test consisted in having the athlete perform a series of sprints with different gears, with and without the device of the invention, so as to evaluate the power and force trends with variations in the RPM, and the maximum power of the athlete.

The results obtained are given in the graph of FIG. 12a in which the power-rate curve obtained with the device of the invention (curve F) and without the device of the invention (curve F′) are given.

It can easily be seen that, with the device of the invention, the power curve has clearly higher values; in particular, the maximum alactacid power produced was greater by +4.45% than the power produced without the device of the invention, and the maximum power produced on average in each sprint was greater by +5.28% than the power produced without the device of the invention.

FIG. 12b gives the force-rate curve obtained with the device of the invention (curve G) and without the device of the invention (curve G′). By analyzing the force curve in the graph of FIG. 12b, it can be seen that, with the device of the invention, there was a considerable increase in the force maxima, for all force expressions: maximum force +6.67%, maximum dynamic force +5.89%, explosive force +3.95%.

Moreover, it can easily be deduced that the device of the invention leads to improvements particularly at low RPM, that is, where there is a need for a greater production of force.

Tests were also carried out to evaluate the effect of the device of the invention on the jumping capacity of some individuals.

A Bosco-Vittori footboard was used for these tests; this measures the height of the jump in centimetres by detecting the flight time, thus evaluating the subject's explosive force.

68 individuals were tested and were divided into 4 homogeneous groups who were required to perform jumps from a standstill with counter movement using a Bosco-Vittori footboard, first without and then with a device of the invention.

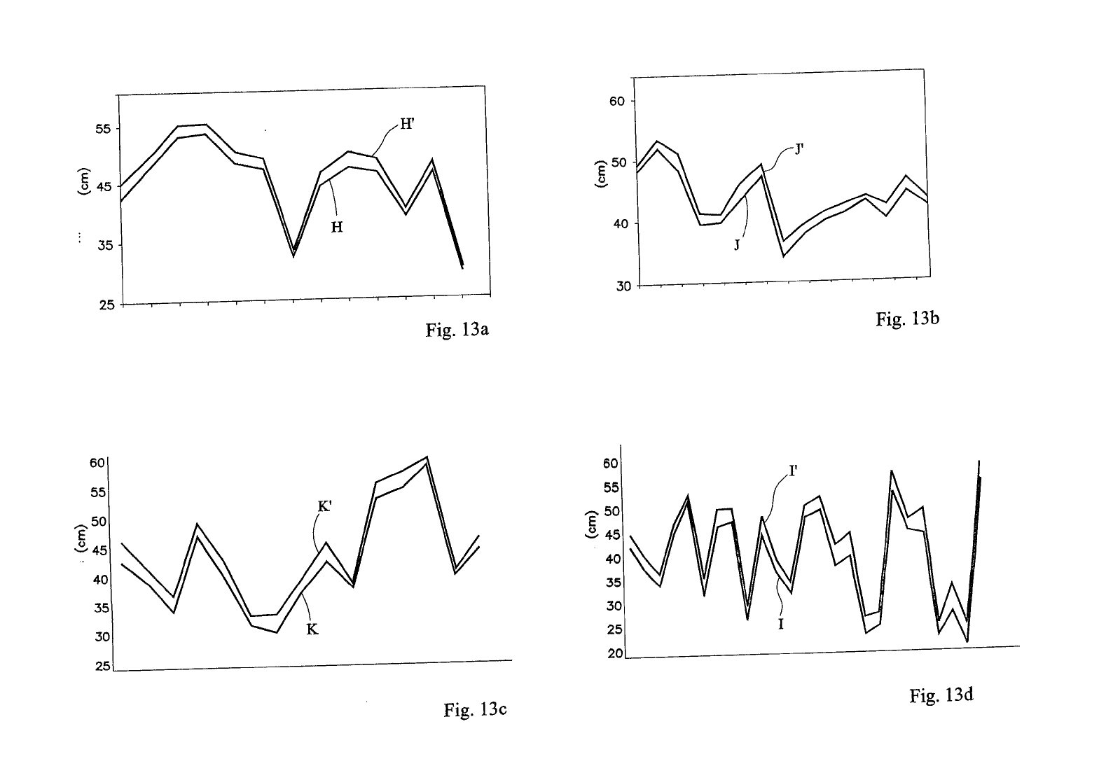

The results are given in the graphs of FIGS. 13a-13d for the following groups of people:

FIG. 13a: 13 female basketball players, curve H without a device, curve H′ with the device of the invention,

FIG. 13b: 15 male basketball players, curve J without a device, curve J′ with the device of the invention;

FIG. 13c: 15 footballers, curve K without a device, curve K′ with the device of the invention,

FIG. 13d: 25 non-sporting subjects aged between 15 and 55 years, curve I without a device, curve I′ with the device of the invention.

It can be seen that, in the test with the devices of the invention, the height of the jumps was greater on average than in the tests without the device of the invention: +3.94% for the group of 13 female basketball players; +4.13% for the 15 male basketball players; +5.56% for the 15 footballers; and an increase on average of +9.26% in the height of the jumps in non-sporting individuals.

Moreover, tests were carried out to evaluate the effect of the device of the invention on an individual's jumping height.

An Optojump® optical measurement system, a device composed of a transmitting bar and of a receiving bar which enable the contact and flight times of the jump to be calculated, was used for these tests; the height of the individual's jump in centimetres and the power of the jumps in Watts/kg was found from these values.

The subject, a male footballer, had to perform a continuous series of jumps on two feet for a period of 30″, first without and then with the device of the invention.

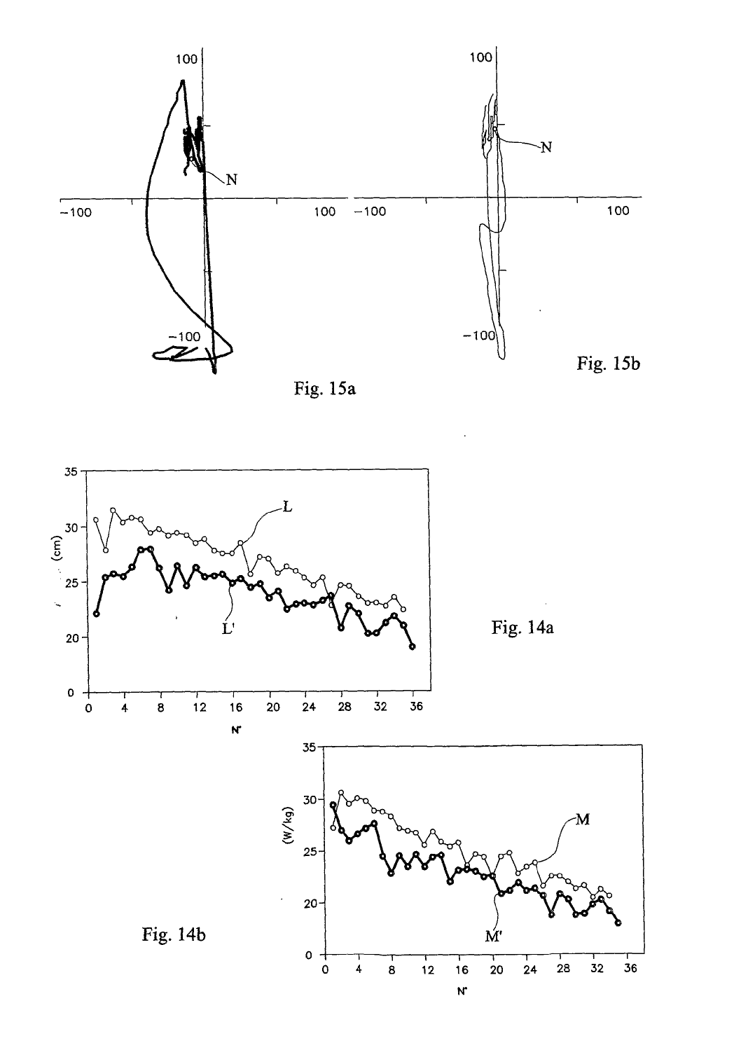

The results obtained are given in the graph of FIG. 14a in which the instantaneous heights (cm) of the jumps within the test period are given with the device of the invention (curve L) and without the device of the invention (curve L′).

It can be seen that, in the test with the device of the invention, the height of the jumps reached by the individual was clearly greater; overall, the average height of the jumps with the device of the invention was greater by 3.02 cm which is equivalent to +12.66%.

The graph of FIG. 14b gives the power produced by the athlete in each jump over the period of the test with the device of the invention (curve M) and without the device of the invention (curve M′).

In this case again, it was found that, as seen previously, in the test with the devices of the invention, the power produced was clearly greater than in the test performed without the device of the invention.

In particular, with the devices of the invention, the individual produced an average power which was greater by 2.38 W/kg, equivalent to +10.45%.

The Applicant has also performed tests to check the effects of the device of the invention on the capacity of an individual to maintain balance during the explosion of a firearm shot.

To perform this test, the TETRAX® posturological system was used, that is, a diagnostic device which analyses the balancing of the subject and the mechanisms which the individual uses to maintain this balance by measurement of the pressure exerted by the individual's feet on two separate platforms.

The individual was placed on a stabilometric platform and fired a series of shots with an 391 Urica 2 rifle.

The tests in question consisted in firing over a period of 20″ with a 391 Urica 2 rifle, first without and then with a device according to the invention; more precisely, for the first 5″ the individual had to stand still in the firing position, he then fired the shot and finally remained in position until the 20″ had elapsed. The results obtained are given in the graphs of FIG. 15a (test without the device of the invention) and 15b (test with the device of the invention), respectively; the moment of firing is indicated by the point N.

In the test performed with the device of the invention, it was noted that the centre of gravity underwent a minimal displacement, a reduction of −19.63% was observed in the displacement of the centre of gravity with a consequent reduction of as much as −77.33% in the area in which the displacement took place.

Finally, the force imparted to the heels during firing (which indicated how the athlete reacted to the recoil of the arm after firing) was less by −25.73% in the firing tests with the device of the invention, with consequent improved support on the front of the feet (+15.06%) and hence greater overall stability of the individual as a whole.

The Applicant has also performed tests to check the effect of the device of the invention on the blood lactate concentration in an individual.

A RUNRACE® Technogym Treadmill and a Lactate Pro® lactacidometer, which measures the blood lactate concentration with the use of reactive strips, were used for the following analysis.

The test consisted in having the individual run for ten minutes at constant speed (14 km/h) first without and, 20 minutes after the end of the first test, with a device of the invention.

In both tests, a lactate sample was taken before the start of the test (basal lactate) and when the test had just finished (lactate at end of test) and the lactate delta which represented the difference between the blood lactic acid concentration at the end and at the beginning of the test, that is, the accumulation of lactic acid, was calculated.

The results of the test were as follows:
1^{st} test without device: basal lactate (before the test): 1.1 m/Mol; lactate at end of test: 8.8 m/Mol; lactate delta: 7.7 m/Mol.
2^{nd} test with the device of the invention: basal lactate (before the test): 1.9 m/Mol; lactate at end of test: 7.0 m/Mol; lactate delta: 5.1 m/Mol.

It can be seen that, in the test with the device of the invention, the blood lactic acid concentration at the end of the test was less by 20.45%, even though the basal lactate was higher.

Overall, in the test carried out with the device of the invention, there was therefore a lower accumulation of lactic acid (−33.77%) in the individual's blood.

The device also enables the individual's organism to tolerate higher training loads, improving his performance.

The device has also been found advantageous for improving the capacity to maintain balance and the stability of an individual close to whose body the above-mentioned device is placed.

This has important consequences not only in the practice of many sports disciplines, for example, cycling, motorcycling, alpinism, sports climbing, etc,. but also in everyday life.

The device is placed in direct or indirect contact with a portion of an individual's skin, for example, by inserting the device in a garment worn by the individual or by the athlete.

To optimize the effects of the device of the invention it is preferably positioned in direct or indirect contact or in the vicinity of the muscles that are used most in the athlete's sports discipline, or incorporated in suitable is garments which will come into contact with those muscles.

For example, the device of the invention may be incorporated in the trousers or the like for athletes in sports in which the work performed by the muscles of the lower half of the body predominates (cyclists, runners, etc), or in the vests in those sports in which the effort exerted by the muscles of the trunk and of the arms predominate (gymnastics, rowing, etc.).

The device may also be positioned in the footwear, either in the inner or in the outer sole, or in the heel thereof.

In other versions, the device may be positioned in helmets or other similar structures for protecting an individual's head or, in particular, an athlete's head.

The device may also be inserted in accessories or garments to be used daily by an individual, improving his physical performance and ability to maintain balance in everyday life.

The device may be incorporated in garments or accessories, even those used for work, or may be associated therewith, in any suitable way, improving an individual's physical performance and ability to maintain balance during the performance of his work tasks.

This has a particular advantage in precise or tiring work in which a consistent physical effort is required or in which potentially dangerous operations are carried out.

The device may optionally also be inserted in a jewel or other accessory such as, for example, a belt, or a glove to be placed close to desired parts of an individual's body to improve his physical performance and ability to maintain balance.

The device 1 is preferably positioned at a distance of less than 3 cm, more preferably less than 2 cm from a portion of the individual's skin in order for the device to be able best to exert its properties on the individual.

The device may also be fitted subcutaneously.

Although the foregoing description refers particularly to sports effects, the device according to the invention may advantageously be used to improve the physical performance of any desired subject and during any activity in which even a minimal muscular effort is required.

The device according to the invention can be prepared in accordance with conventional methods for the preparation of multi-layered metallic bodies by the application of a layer of insulating material to the metal body obtained, by means of any suitable known method.

A method of preparing a device according to the invention provides of the provision of a metal body suitable for constituting the inner layer 4, 4′ of the device 1, 1′ according to one of the versions described above, and subsequent glazing and polishing of the metal body and ultrasound degreasing at about 60° C. for about 5-7 minutes.

Washing is then performed to eliminate undesired compounds followed by further washing in hot distilled water at about 60° C. Electrolytic degreasing is then performed at ambient temperature with a voltage of between 6 and 10 volts for about one minute, followed by a series of hot washes at about 60° C. with distilled water.

The metal body is then subjected to neutralization at ambient temperature by immersion for about 20-30 seconds and is then subjected to a further washing with hot distilled water at about 60° C. Rhodium plating follows at a temperature of between about 40° C. and 50° C., preferably at about 45° C. with the use of galvanic-bath rhodium at a concentration of between 1 and 8 g/l at about 50° C., the metal body being left in contact with the rhodium solution for a period of time of between about 2 and about 10 seconds, according to the thickness of rhodium to be obtained.

The metal body 2 is left in contact with the rhodium solution for a maximum period of 10 minutes.

If a considerable thickness of rhodium of about 5 μm is to be obtained, the step of the deposition of the rhodium layer is performed in several successive stages, that is, the body is immersed in the galvanic bath for a predetermined period of time, removed from the bath, subjected to washings and degreasings, and immersed in the bath again.

This series of steps is repeated a desired number of times, according to the final thickness to be obtained.

This enables a layer of rhodium to be obtained which adheres to the metal body, eliminating the known problems of detachment of rhodium layers of considerable thickness.

This also prevents blackening of the rhodium layer.

The metal body is then subjected to washing in distilled water.

If further layers are required in the device 1, they are applied by suitable techniques.

The insulating covering layer is then applied.

1. A device for improving an individual's physical performance and ability to maintain balance, the device being suitable for being placed close to a portion of the individual's skin and comprising at least one metal body which is provided with a first metal layer, at least one second metal layer that is based on a metal selected from Ruthenium, Rhodium and Platinum and is at least partially superimposed on the first layer, and an insulating layer which at least partially covers the metal body.

2. The device according to claim 1, wherein in which the first layer is based on a metal selected from the group comprising Silver, Gold, Platinum, Copper, Aluminium and steel.

3. The device according to claim 1, wherein the second layer is at least partially superimposed on both opposed main surfaces of the first layer.

4. The device according to claim 1, wherein the second metal layer is based on Rhodium having a thickness of about 2-6 μm.

5. The device according to claim 1, wherein the second metal layer is based on Ruthenium or Platinum and the metal body further comprises a third, Rhodium-based layer which is associated with the second layer in a manner such that the second layer is interposed between the first layer and the third layer.

6. The device according to claim 5, wherein the second metal layer is based on Platinum and the metal body further comprises an intermediate, Ruthenium-based layer interposed between the first layer and the third layer.

7. The device according to claim 1, wherein the insulating layer completely covers the metal body.

8. The device according to claim 1, wherein the insulating layer comprises a plastics material selected from a group comprising: PVC, polyurethane (PU), nylon, polypropylene, polyethylene (PE), high-density polyethylene (HDPE), PET or fibres derived therefrom, PTFE, and silicone rubbers.

9. The device according to claim 1, wherein the insulating layer comprises an insulating material of natural origin selected from a group comprising rubber, natural gum, latex, or a natural insulating fibre, for example, wool, cotton, and hemp.

10. The device according to claim 1, wherein the insulating layer is shaped in a manner such as to seal the metal body from the exterior.

11. The device according to claim 1, wherein the insulating layer is fitted in a manner such that the metal body is under vacuum.

12. A garment in a portion of which at least one device according to claim 1 is fitted or incorporated.

13. Footwear in a portion of which at least one device according to claim 1 is fitted and/or incorporated.

14. The footwear according to claim 13, wherein the at least one device is fitted inside the footwear in the region of a sole thereof.

15. A helmet comprising an inner surface on a portion of which at least one device according to claim 1 is fitted and/or incorporated.

16. A jewel in which at least one device according to claim 1 is fitted and/or incorporated.

17. A method of using a device for improving an individual's physical performance, comprising the steps of:
providing a device according to claim 1, and
placing the device close to a portion of an individual's skin, or
implanting the device under a portion of an individual's skin
